(19) United States
(12) Patent Application Publication
(10) Pub. No.: US 2008/0145409 A1
(43) Pub. Date: Jun. 19, 2008

(54) COMPOSITION FOR THE TREATMENT AND PREVENTION OF PEPTIC ULCER

(76) Inventors: Min Chang Huang, Taipei County (TW); Guang-Tzuu Shane, Taipei County (TW); Chang-Hua Yang, Taipei County (TW); Kuo-Yen Chen, Taipei County (TW)

Correspondence Address: BAKER & MCKENZIE LLP, Pennzoil Place, South Tower, 711 Louisiana, Suite 3400, HOUSTON, TX, 77002-2716, US

(21) Appl. No.: 11/612,549

(22) Filed: Dec. 19, 2006

(51) Int. Cl. A61K 31/045; A61K 31/11; A61K 31/201; A61K 47/46; A61P 1/04

(52) U.S. Cl. 424/439; 514/739; 514/703; 514/557

(57) ABSTRACT

The present invention relates to a composition and methods of administering the composition, comprising Citronellol and its analogues and derivatives, to humans and other mammalian animals with peptic ulcers induced by alcohol consumption, H. pylori infection, stress and/or intake of nonsteroidal anti-inflammatory medications.

### FIELD OF THE INVENTION

The invention relates to a composition and the method of administering such composition, comprising Citronellol, Citronellol analogues and/or derivatives, for the prevention and treatment of peptic ulcers in mammals.

### BACKGROUND OF THE INVENTION

Peptic ulcers are erosions of mucous membranes in the lower part of the esophagus, the stomach, the duodenum, and the jejunum. The most common forms of peptic ulcers are duodenal and gastric ulcers. Peptic ulcers are generally caused by an imbalance between the secretion of acid, pepsin and the defenses of the stomach's or duodenum's mucosal lining. In particular, neutrophils are known to release several reactive oxidation intermediates (ROI), such as O₂⁻, H₂O₂, that can lead to the imbalance between acid secretion/pepsin and the defense mechanism. Some of the risk factors for peptic ulcers include: stress, use of nonsteroidal anti-inflammatory medications (NSAIDs) such as aspirin, smoking, alcohol consumption and Helicobacter Pylori bacterial infection. Infection with H. pylori has been found to be the cause of 90% of duodenal ulcers and 80% of gastric ulcers.

H. pylori is a spiral shaped gram-negative bacterium that lives in the mucous tissues that line the digestive tract. For people with H. pylori infection, the main goal is eradication of the organism that causes the problem. Multiple regimens are effective and usually include either an H2 receptor antagonist such as famotidine (Pepcid) or nizatidine (Axid) or a proton pump inhibitor such as omeprazole (Prilosec) or esomeprazole (Nexium) to suppress acid, combined with antibiotics. However, such a treatment plan relies heavily on the use of antibiotics and involves the administration of a combination of drugs.

It has also been documented that some essential oils, including geranium and citronella oils, have in vitro anti-inflammatory effect. Specifically, it is shown that some essential oils have inhibitory activities on the adherence reaction of human peripheral neutrophils induced by tumor necrosis factor-alpha (TNF-α). Other studies suggest that cutaneous application of geranium essential oil has the suppressive activity of neutrophil accumulation in mice. However, it remains unknown whether these essential oils would be useful in the treatment or prevention of ulcers. Thus, the inventors are led to explore and experiment the therapeutic effect of Citronellol and its analogues/derivatives.

### SUMMARY OF THE INVENTION

As used herein, Citronellol derivatives include, but not limited to, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, and (−)-β-Citronellol. Citronellol analogues include, but not limited to, geraniol.

It is an object of the present invention to provide a composition for the treatment or prevention of peptic ulcer in mammals. The composition comprises Citronellol, Citronellol analogues and/or derivatives. The causes of the peptic ulcer include, but not limited to, alcohol consumption, Helicobacter pylori bacterial infection, stress, and intake of NSAIDs. The composition can be administered orally, through intravenous or intraperitoneal injection, or through other medically acceptable routes. The form of the composition is not limited as long as it can perform the desired therapeutic function. Preferably, the composition is prepared in powder, particle, capsule, tablet, injectable perfusion, oral solution, oral suspension, or other pharmaceutically acceptable forms.

It is another object of the present invention to provide a pharmaceutical formulation for the treatment or prevention of peptic ulcer in mammals. The pharmaceutical formulation comprises an effective amount of Citronellol, Citronellol analogues and/or derivatives, together with a pharmaceutically acceptable carrier, diluent or excipient. The effective amount of Citronellol, Citronellol analogues and/or derivatives is not limited, as long as it is effective for the treatment or prevention of peptic ulcer. Preferably, the effective amount of Citronellol, Citronellol analogues and/or derivatives ranges from 0.5 mg/kg to 50 mg/kg.

It is another object of the present invention to provide a method for treating or preventing peptic ulcer in mammals. The method comprises: administering to a subject a composition comprising Citronellol, Citronellol analogues and/or derivatives. Note that the administering route is not limited, as long as the active ingredient can be effectively absorbed without undesired side effect. Preferably, the routes of administration include oral, intravenous injection, intraperitoneal injection, and cutaneous application.

It is another object of the present invention to provide a use of Citronellol, Citronellol analogues and/or derivatives in the preparation of a pharmaceutical agent, supplement, food, or food ingredient for the treatment or prevention of peptic ulcer. Note that the form of final product is not limited, as long as Citronellol analogues and/or derivatives can be effectively absorbed (or [symbol] ) without undesired side effect.

The foregoing and other objects, features and advantages of the present invention will become more apparent from the following detailed description when taken in conjunction with the accompanying drawings.

### DESCRIPTION OF EMBODIMENTS OF THE INVENTION

In order to validate the therapeutic effect of the composition of the present invention that comprises Citronellol, Citronellol analogues and/or derivatives, two sets of experiments were designed and carried out. The first set of experiments was directed to evaluate the dose-dependent therapeutic effect of the composition on gastric ulcer induced by alcohol consumption. The second set of experiments was directed to gastric ulcer induced by H. pylori bacteria infection. Both experiments will be discussed in detail below.

Note that the dosage used in each of the experiments is considered as exemplary only and shall not be construed as limiting the effective dosage to any particular range. Further, as known in the art, the route of administration of the composition shall not be limited as long as the intended therapeutic effect can be achieved. All medically acceptable procedures, such as intravenous injection, intraperitoneal injection, oral intake and the like, can be used in the present invention.

Unless defined otherwise, the meanings of all technical and scientific terms used herein are those commonly understood by one of ordinary skill in the art to which this invention belongs. One skilled in the art will also appreciate that any methods and materials similar or equivalent to those described herein can also be used to practice or test the invention.

Moreover, all numbers expressing quantities of ingredients, reaction conditions, % purity, and etc., used in the specification and claims, are modified by the term “about,” unless otherwise indicated. Accordingly, the numerical parameters set forth in the specification and claims are approximations that may vary depending upon the desired properties of the present invention.

The following examples illustrate the present invention. They are merely exemplary and shall not be construed as limiting the invention.

### EXAMPLE 1

### Citronellol and its Analogues/Derivatives in the Treatment and Prevention of Gastric Ulcer Induced by Alcohol Consumption

The purpose of this study was to investigate the effect of MIC31 (Citronellol) and its analogues/derivatives on gastric injury induced by ethanol in the rat. Gastric damage was produced by oral administration of absolute ethanol to rats. The severity of the ethanol-induced gastric damage varied considerably within the vehicle-treated group of rats which served as the negative controls.

Animals studied in this example were Sprague-Dawley derived Male Rats from Yung-Min Medical University Laboratory Animal Center. The age of these rats are 7 weeks old. Each group has 5 rats, and their body weight at arrival was 180±10 gm. Upon arrival, heath status of rats will undergo a minimum of one week acclimation period prior to the start of the experiment. At the first day of study, body weights are measured and the animals are grouped according to experiment design. The environmental conditions are listed below:

Temperature: 22° C.-24° C.

Relative humidity 60%-70%

Light cycle 12 hour dark/12 hour light (lights on at ca 7:00 A.M.)

Diet: Lab Diet, Rodent Diet

Quantity: Ad libitum

Water: Ad libitum

The compounds tested in this example are listed in Table 1:

TABLE 1
Compounds tested in Example 1

| Compound number | Compound name |
|---|---|
| MIC-31 | Citronellol, purchased from Taipei, Taiwan Fine |
| | chemicals Co., Ltd. |
| MIC-32 | Geraniol, purchased from Taipei, Taiwan Fine |
| | chemicals Co., Ltd.^{†} |
| MIC-33 | Geraniol, FL-48798 |
| MIC-34 | Citronellal, SI-C2513* |
| MIC-35 | Citronellic acid, AL-303429* |
| MIC-36 | (s)-(+)-Citronellyl bromide, AL-377716* |
| MIC-38 | Citronellyl isobutryrate, AL-231304* |
| MIC-39 | Citronellyl acetate, AL-W231118* |
| MIC-40 | Citronellyl propionate, AL-W231606* |
| MIC-41 | Citronellyl formate, AL-W231401* |
| MIC-42 | Citronellol, AL-W230901* |
| MIC-43 | (R)-(−)-Citronellyl bromide, AL-377392* |
| MIC-44 | Citronellyl tiglate, AL-W500607* |
| MIC-45 | (−)-β-Citronellol, FL-27483* |

*Except for MIC-32, all Citronellol analogues were purchase from Sigma Chemical Co., St. Louis, MO
^{†}MIC-32 was used in Example 2.

Note that MIC-31 and MIC-42 are actually the same but purchased from different sources. MIC-32 and MIC-33 are also the same but purchased from different sources.

The experimental procedure is described below:

### Gastric ulcers Ethanol

Test substance MIC31 and its analogues/derivatives at doses 300 mg/kg, 100 mg/kg or 30 mg/kg and vehicle (2% Tween 80) and the positive control Carbenoxolone (300 mg/kg) are administered P.O. (10 ml/kg) to a group of 5 Sprague-Dawley derived male rats overnight fasted weighing 180±10 gm at 30 minutes before absolute ethanol challenge (1 ml/rat, P.O.). One hour later, the animals are sacrificed and the stomachs are opened along the greater curvature. Gastric ulceration is scored for degree of hemorrhage and severity of ulcerative lesions as follows: 0=no hyperemia or lesion (dark red blood clot), 1=hyperemia, 2=one or two slight lesions, 3=more than two slight lesions, 4=more than two lesions or severe lesions. Reduction of concurrent control score values by 50 percent or more (≧50%) is considered significant. During the experimental phase, body weight will be documented.

All the Citronellol and its analogues/derivatives were tested following the design listed in Table 2:

TABLE 2
Experimental design of Example 1

| | The dosing and administration | | | | | | | |
|---|---|---|---|---|---|---|---|---|
| | | | | | Dosage | | | |
| Test | Group | Test | Route | Conc. mg/ml | ml/kg | mg/kg | rats | P.O. with MIC analogues |
| 1 | 1 | Vehicle | P.O. | (2% Tween80) | 10 | NA | 5 | 30 mins Before challenge |
| | 2 | Omeprazole | P.O. | 5 mg/ml | 10 | 50 mg/kg | 5 | 30 mins Before challenge |
| | 3 | MIC-31 | P.O. | 10 mg/ml | 10 | 100 mg/kg | 5 | 30 mins Before challenge |
| | 4 | MIC-31 | P.O. | 3 mg/ml | 10 | 30 mg/kg | 5 | 30 mins Before challenge |
| 2 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-33 | P.O. | 30 mg/ml | 10 | 300 mg/kg | 5 | 30 mins Before challenge |
| | 4 | MIC-33 | P.O. | 10 mg/ml | 10 | 100 mg/kg | 5 | 30 mins Before challenge |
| | 5 | MIC-33 | P.O. | 3 mg/ml | 10 | 30 mg/kg | 5 | 30 mins Before challenge |
| 3 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-34 | P.O. | 30 mg/ml | 10 | 300 mg/kg | 5 | 30 mins Before challenge |
| | 4 | MIC-34 | P.O. | 10 mg/ml | 10 | 100 mg/kg | 5 | 30 mins Before challenge |
| | 5 | MIC-34 | P.O. | 3 mg/ml | 10 | 30 mg/kg | 5 | 30 mins Before challenge |
| 4 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-35 | P.O. | 30 mg/ml | 10 | 300 mg/kg | 5 | 30 mins Before challenge |
| | 4 | MIC-35 | P.O. | 10 mg/ml | 10 | 100 mg/kg | 5 | 30 mins Before challenge |
| | 5 | MIC-35 | P.O. | 3 mg/ml | 10 | 30 mg/kg | 5 | 30 mins Before challenge |
| 5 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-36 | P.O. | 30 mg/ml | 10 | 300 mg/kg | 5 | 30 mins Before challenge |
| | 4 | MIC-36 | P.O. | 10 mg/ml | 10 | 100 mg/kg | 5 | 30 mins Before challenge |
| | 5 | MIC-36 | P.O. | 3 mg/ml | 10 | 30 mg/kg | 5 | 30 mins Before challenge |
| 6 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-38 | P.O. | 30 mg/ml | 10 | 300 mg/kg | 5 | 30 mins Before challenge |
| | 4 | MIC-38 | P.O. | 10 mg/ml | 10 | 100 mg/kg | 5 | 30 mins Before challenge |
| | 5 | MIC-38 | P.O. | 3 mg/ml | 10 | 30 mg/kg | 5 | 30 mins Before challenge |
| 7 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-39 | P.O. | 30 mg/ml | 10 | 300 mg/kg | 5 | 30 mins Before challenge |
| | 4 | MIC-39 | P.O. | 10 mg/ml | 10 | 100 mg/kg | 5 | 30 mins Before challenge |
| | 5 | MIC-39 | P.O. | 3 mg/ml | 10 | 30 mg/kg | 5 | 30 mins Before challenge |
| 8 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-40 | P.O. | 30 mg/ml | 10 | 300 mg/kg | 5 | 30 mins Before challenge |
| | 4 | MIC-40 | P.O. | 10 mg/ml | 10 | 100 mg/kg | 5 | 30 mins Before challenge |
| | 5 | MIC-40 | P.O. | 3 mg/ml | 10 | 30 mg/kg | 5 | 30 mins Before challenge |
| 9 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-41 | P.O. | 30 mg/ml | 10 | 300 mg/kg | 5 | 30 mins Before challenge |
| | 4 | MIC-41 | P.O. | 10 mg/ml | 10 | 100 mg/kg | 5 | 30 mins Before challenge |
| | 5 | MIC-41 | P.O. | 3 mg/ml | 10 | 30 mg/kg | 5 | 30 mins Before challenge |
| 10 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-42 | P.O. | 30 mg/ml | 10 | 300 mg/kg | 5 | 30 mins Before challenge |
| | 4 | MIC-42 | P.O. | 10 mg/ml | 10 | 100 mg/kg | 5 | 30 mins Before challenge |
| | 5 | MIC-42 | P.O. | 3 mg/ml | 10 | 30 mg/kg | 5 | 30 mins Before challenge |
| 11 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-43 | P.O. | 30 mg/ml | 10 | 300 mg/kg | 5 | 30 mins Before challenge |
| | 4 | MIC-43 | P.O. | 10 mg/ml | 10 | 100 mg/kg | 5 | 30 mins Before challenge |
| | 5 | MIC-43 | P.O. | 3 mg/ml | 10 | 30 mg/kg | 5 | 30 mins Before challenge |
| 12 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-44 | P.O. | 30 mg/ml | 10 | 300 mg/kg | 5 | 30 mins Before challenge |
| | 4 | MIC-44 | P.O. | 10 mg/ml | 10 | 100 mg/kg | 5 | 30 mins Before challenge |
| | 5 | MIC-44 | P.O. | 3 mg/ml | 10 | 30 mg/kg | 5 | 30 mins Before challenge |
| 13 | 1 | Vehicle | P.O. | (2% Tween80) | 10 | NA | 5 | 30 mins Before challenge |
| | 2 | Carbenoxolone | P.O. | 30 mg/ml | 10 | 300 mg/kg | 5 | 30 mins Before challenge |
| | 3 | MIC-45 | P.O. | 30 mg/ml | 10 | 300 mg/kg | 5 | 30 mins Before challenge |
| | 4 | MIC-45 | P.O. | 10 mg/ml | 10 | 100 mg/kg | 5 | 30 mins Before challenge |
| | 5 | MIC-45 | P.O. | 3 mg/ml | 10 | 30 mg/kg | 5 | 30 mins Before challenge |

The results of all the tested compound in different dosage are listed in the following Table 3:

TABLE 3
Result of Example 1

| Test | Group | compound | Score (0, 1, 2, 3, 4) | Average | Reduction (%) |
|---|---|---|---|---|---|
| 1 | 1 | Vehicle | 3, 3, 4, 4, 4 | 3.6 | 0% |
| | 2 | Omeprazole 50 mg/kg | 0, 4, 3, 4, 0 | 2.2 | 39% |
| | 3 | MIC-31 100 mg/kg | 0, 0, 0, 1, 1 | 0.4 | 89% |
| | 4 | MIC-31 30 mg/kg | 0, 0, 2, 3, 0 | 1 | 72% |
| 2 | 1 | Vehicle | 4, 4, 4, 2, 3 | 3.4 | 0% |
| | 2 | Carbenoxolone 300 mg/kg | 0, 2, 1, 1, 1 | 1 | 71% |
| | 3 | MIC-33 300 mg/kg | 0, 2, 1, 1, 1 | 1 | 71% |
| | 4 | MIC-33 100 mg/kg | 0, 1, 1, 1, 1 | 0.8 | 76% |
| | 5 | MIC-33 30 mg/kg | 0, 1, 2, 2, 2 | 1.4 | 59% |
| 3 | 1 | Vehicle | 4, 4, 4, 3, x | 3.8 | 0% |
| | 2 | Carbenoxolone 300 mg/kg | 3, 1, 0, 2, 0 | 1.2 | 68% |
| | 3 | MIC-34 300 mg/kg | 2, 2, 2, 2, 0 | 1.6 | 58% |
| | 4 | MIC-34 100 mg/kg | 0, 2, 1, 0, 2 | 1 | 74% |
| | 5 | MIC-34 30 mg/kg | 2, 3, 1, 0, 1 | 1.4 | 63% |
| 4 | 1 | Vehicle | 2, 4, 3, 4, 3 | 3.2 | 0% |
| | 2 | Carbenoxolone 300 mg/kg | 1, 3, 2, 0, 0 | 1.2 | 63% |
| | 3 | MIC-35 300 mg/kg | 1, 0, 3, 0, 3 | 1.4 | 56% |
| | 4 | MIC-35 100 mg/kg | 1, 1, 0, 3, 3, | 1.6, | 50% |
| | 5 | MIC-35 30 mg/kg | 4, 1, 1, 1, 3 | 2 | 38% |
| 5 | 1 | Vehicle | 3, 3, 1, 4, 4 | 3 | 0% |
| | 2 | Carbenoxolone 300 mg/kg | 0, 1, 3, 0, 3 | 1 | 53% |
| | 3 | MIC-36 300 mg/kg | 1, 2, 0, 2, 3 | 1.6 | 47% |
| | 4 | MIC-36 100 mg/kg | 1, 2, 2, 1, 1 | 1.4 | 53% |
| | 5 | MIC-36 30 mg/kg | 2, 0, 3, 3 | 2 | 33% |
| 6 | 1 | Vehicle | 2, 3, 2, 4, 4 | 3 | 0% |
| | 2 | Carbenoxolone 300 mg/kg | 1, 2, 2, 1, 0 | 1.2 | 60% |
| | 3 | MIC-38 300 mg/kg | 1, 0, 1, 3, 1 | 1.2 | 60% |
| | 4 | MIC-38 100 mg/kg | 1, 2, 3, 2, 1 | 1.8 | 40% |
| | 5 | MIC-38 30 mg/kg | 3, 4, 2, 4, 3 | 3.2 | 0% |
| 7 | 1 | Vehicle | 3, 3, 2, 3, 4 | 3 | 0% |
| | 2 | Carbenoxolone 300 mg/kg | 2, 0, 0, 0, 0 | 0.4 | 87% |
| | 3 | MIC-39 300 mg/kg | 0, 4, 0, 0, 0, | 0.8 | 73% |
| | 4 | MIC-39 100 mg/kg | 0, 0, 3, 1, 0 | 0.8 | 73% |
| | 5 | MIC-39 30 mg/kg | 2, 2, 2, 3, 3 | 2.4 | 20% |
| 8 | 1 | Vehicle | 3, 3, 4, 4, 4 | 3.6 | 0% |
| | 2 | Carbenoxolone 300 mg/kg | 0, 0, 0, 0, 2 | 0.4 | 89% |
| | 3 | MIC-40 300 mg/kg | 3, 0, 0, 03 | 1.2 | 67% |
| | 4 | MIC-40 100 mg/kg | 3, 2, 3, 2, 4 | 2.8 | 22% |
| | 5 | MIC-40 30 mg/kg | 3, 4, 4, 4, 2 | 3.4 | 6% |
| 9 | 1 | Vehicle | 3, 4, 4, 3, 2 | 3.2 | 0% |
| | 2 | Carbenoxolone 300 mg/kg | 1, 1, 1, 3, 1 | 1.4 | 56% |
| | 3 | MIC-41 300 mg/kg | 1, 2, 1, 3, 0 | 1.4 | 56% |
| | 4 | MIC-41 100 mg/kg | 0, 1, 2, 2, 0 | 1 | 69% |
| | 5 | MIC-41 30 mg/kg | 4, 4, 3, 1, 4 | 3.2 | 0% |
| 10 | 1 | Vehicle | 4, 4, 2, 3, 4 | 3.4 | 0% |
| | 2 | Carbenoxolone 300 mg/kg | 0, 0, 2, 2, 2 | 1.2 | 65% |
| | 3 | MIC-42 300 mg/kg | 1, 2, 2, 0, 3 | 1.6 | 53% |
| | 4 | MIC-42 100 mg/kg | 0, 0, 2, 0, 2 | 0.8 | 76% |
| | 5 | MIC-42 30 mg/kg | 2, 2, 2, 0, 2 | 1.6 | 53% |
| 11 | 1 | Vehicle | 4, 4, 2, 3, 4 | 3.4 | 0% |
| | 2 | Carbenoxolone 300 mg/kg | 0, 0, 2, 2, 2 | 1.2 | 65% |
| | 3 | MIC-43 300 mg/kg | 2, 2, 0, 2, 2 | 1.6 | 53% |
| | 4 | MIC-43 100 mg/kg | 2, 3, 1, 2, 0 | 1.6 | 53% |
| | 5 | MIC-43 30 mg/kg | 2, 3, 2, 2, 3 | 2.4 | 29% |
| 12 | 1 | Vehicle | 3, 4, 4, 4, 3 | 3.6 | 0% |
| | 2 | Carbenoxolone 300 mg/kg | 0, 0, 1, 0, 1 | 0.4 | 89% |
| | 3 | MIC-44 300 mg/kg | 3, 4, 2, 2, 0 | 2.2 | 39% |
| | 4 | MIC-44 100 mg/kg | 4, 1, 4, 0, 0 | 1.8 | 50% |
| | 5 | MIC-44 30 mg/kg | 0, 4, 4, 2, 4 | 2.8 | 22% |
| 13 | 1 | Vehicle | 3, 4, 4, 4, 3 | 3.6 | 0% |
| | 2 | Carbenoxolone 300 mg/kg | 0, 0, 1, 0, 1 | 0.4 | 89% |
| | 3 | MIC-45 300 mg/kg | 1, 0, 1, 0, 1 | 1.6 | 83% |
| | 4 | MIC-45 100 mg/kg | 1, 2, 0, 2, 1 | 1.2 | 67% |
| | 5 | MIC-45 30 mg/kg | 0, 3, 2, 4, 4 | 2.6 | 28% |

According to the results of Example 1, MIC31 (Citronellol) can inhibit the severity degree of hemorrhage and ulcerative lesions in stomach after the challenge with absolute ethanol. In order to evaluate whether other MIC31 analogue/derivative has similar protecting effect on the stomach, ten MIC31 analogues/derivatives were also tested under the same condition. The testing results clearly show that most of the Citronellol analogues/derivatives have similar protecting effect to stomach after challenge with absolute ethanol. The results show that MIC33 (Geranol) and MIC34 (Citronellal) offer greater protection than MIC31 (Citronellol). The other MIC31 analogues/derivatives show very similar effect as MIC31. The evidence indicates that MIC31 and its analogues/derivatives could efficiently inhibit the severity degree of hemorrhage and ulcerative lesions in stomach after challenge with absolute ethanol. In conclusion, Citronellol and its analogues/derivatives, administered orally as a single dose 30 min before alcohol challenge, significantly decreased the degree of developed severe lesions. These results suggest that MIC31 and its analogues/derivatives were effective in increasing the resistance of the gastric mucosa to ethanol. Note that although each compound was tested as a single active ingredient, the combination of two or more Citronellol analogues/derivatives also have similar therapeutic/prevention effects. Therefore, the composition of the present invention can comprise more than one kind of Citronellol analogues/derivatives in order to achieve a better result on combating gastric ulcer induced by alcohol consumption.

### EXAMPLE 2

### Citronellol and its Analogue in the Treatment/Prevention of Gastric Ulcer Induced by H. pylori Bacteria Infection

MIC-31 (Citronellol) and MIC-32 (Geraniol) were evaluated for their abilities to protect mice from the Helicobacter pylori-induced ulcers. MIC-31 was dosed at 50, 25, 12.5 and 6.25 mg/kg, P.O., as well as at 25 and 12.5 mg/kg, I.P. MIC-32 was dosed at 25 and 12.5 mg/kg, P.O. These dosing regimes were administered twice daily for 7 consecutive days beginning with the first dose given at one hour after Helicobacter pylori inoculation. On day 8, Gastric ulceration was scored as 0, 1, 2 or 3 (3 being most severe relative to vehicle control) according to the degree of hemorrhage and severity of lesions on the gastric mucosa.

The experimental procedures is described below:

### Test Substance and Dosing Patterns:

MIC-31 and MIC-32 were dissolved in 2% Tween 80 for oral (P.O.) and in 2% Tween 80/0.9% NaCl for intraperitoneal (I.P.) administration. MIC-31 at doses of 50, 25, 12.5, 6.25 mg/kg (P.O.) and 25, 12.5 mg/kg (I.P.), as well as MIC-32 at 25 and 12.5 mg/kg (P.O.), were administered to test animals twice daily for 7 consecutive days. The dosing volume was 10 ml/kg.

### Animals:

Male CD-1 (Crl.) derived mice weighing 24±2 g were provided. Space allocation for 10 animals was 29×18×13 cm. Mice were housed in cages and maintained in a controlled temperature (22° C.-23° C.) and humidity (70%-80%) environment with 12 hours light dark cycles for at least one week prior to use. Free access to standard lab chow for mice and tap water was granted.

### Methods:

Groups of 5 male CD-1 (Crl.) derived mice weighing 24±2 g, were fasted for 18 hours prior to intragastric inoculation of Helicobacter pylori in suspension at 9.5×10⁹ CFU/0.4 ml/mouse. MIC-31 at 50, 25, 12.5 and 6.25 mg/kg, MIC-32 at 25 and 12.5 mg/kg and vehicle (2% Tween 80, 10 ml/kg) were each administered orally to test animals, starting one hour after the Helicobacter pylori inoculation, dosing twice daily (9:00 A.M. and 16:00 P.M.) for 7 consecutive days. MIC-31 was also dosed intraperitoneally at 25 mg/kg and 12.5 mg/kg, starting also one hour after the Helicobacter pylori inoculation, twice daily for 7 consecutive days. Omeprazole 1 mg/kg and Clarithromycin 10 mg/kg, in combination, were used as positive controls and administered orally to test animals once daily for 7 consecutive days under the same treatment regime. Eight days after infection, all animals were fasted overnight and sacrificed. Each stomach was dissected along the greater curvature. Gastric ulceration was scored at four levels according to the degree of hemorrhage and severity of ulcerative lesions: 0=normal appearance, 1=mild red spots, 2=moderate red spots and/or hemorrhage spots, 3=marked hemorrhage spots. Reduction of ulceration score by 50 percent or more (≧50%) relative to vehicle control score values is considered significant. In addition, blood of each animal was collected from the retro-orbital sinus on day 8 and plasma sample was kept frozen at −80° C. until returned to the sponsor. Tissues of stomach and intestines were removed by surgical excision and immersed in 10% Neutral-Buffer formalin for histopathological examination.

The results of Example 2 are listed in Table 4 and 5.

TABLE 4
Experimental results of Example 2 through P.O. route

| | | | | | Ulceration Score | | | | | | |
|---|---|---|---|---|---|---|---|---|---|---|---|
| | | | | | Individual | | | | | | |
| Treatment | Route | Dose | | N | 1 | 2 | 3 | 4 | 5 | Total | % Inhibition |
| Vehicle | PO | 10 | ml/kg × 2 × 7 | 5 | 3 | 3 | 3 | 3 | 3 | 15 | — |
| (2% Tween 80) | | | | | | | | | | | |
| PT# 1059525-ADD | PO | 50 | mg/kg × 2 × 7 | 5 | 1 | 1 | 0 | 0 | 0 | 2 | (87) |
| (MIC-31) | | | | | | | | | | | |
| | PO | 25 | mg/kg × 2 × 7 | 5 | 1 | 0 | 0 | 1 | 1 | 3 | (80) |
| | PO | 12.5 | mg/kg × 2 × 7 | 5 | 3 | 0 | 1 | 1 | 2 | 7 | (53) |
| | PO | 6.25 | mg/kg × 2 × 7 | 5 | 1 | 1 | 3 | 3 | 3 | 11 | 27 |
| PT# 1059526-ADD | PO | 25 | mg/kg × 2 × 7 | 5 | 2 | 3 | 2 | 1 | 3 | 11 | 27 |
| (MIC-32) | | | | | | | | | | | |
| | PO | 12.5 | mg/kg × 2 × 7 | 5 | 3 | 1 | 3 | 3 | 3 | 13 | 13 |
| Omeprazole + Clarithromycin | PO | (1 + 10) | mg/kg × 7 | 5 | 1 | 0 | 0 | 1 | 1 | 3 | (80) |

Test substances and vehicle control (2% Tween 80) were each administered orally to test animals twice daily for 7 consecutive days. The Heticobacter pylori (9.5 × 10⁹ CFU/0.4 ml/mouse) inoculation was applied one hour before the first dose. All overnight-fasted animals were sacrificed on day 8. Each stomach was dissected along greater curvature. Reduction of ulceration score by 50 percent or more (≧50%) relative to vehicle control score values isconsidered significant.

TABLE 5
Experimental results of Example 2 through I.P. route

| | | | | | Ulceration Score | | | | | | |
|---|---|---|---|---|---|---|---|---|---|---|---|
| | | | | | Individual | | | | | | |
| Treatment | Route | Dose | | N | 1 | 2 | 3 | 4 | 5 | Total | % Inhibition |
| Vehicle | IP | 10 | ml/kg × 2 × 7 | 5 | 3 | 3 | 3 | 3 | 3 | 15 | — |
| (2% Tween 80/ | | | | | | | | | | | |
| 0.9% MaCl) | | | | | | | | | | | |
| PT# 1059525-ADD | IP | 25 | mg/kg × 2 × 7 | 5 | 1 | 2 | 3 | 0 | 2 | 8 | 47 |
| (MIC-31) | | | | | | | | | | | |
| | IP | 12.5 | mg/kg × 2 × 7 | 5 | 0 | 0 | 0 | 0 | 2 | 2 | (87) |
| Omeprezole + Clarithromycin | PO | (1 + 10) | mg/kg × 7 | 5 | 2 | 0 | 2 | 1 | 0 | 5 | (67) |

Test substances and vehicle control (2% Tween 80/0.9% NaCl) were each administered intraperitoneally to test animals twice daily for 7 consecutive days. The Helicobacter pylori (9.5 × 10⁹ CFU/0.4 ml/mouse) inoculation was applied one hour befor the first dosing. All overnight-fasted animals were sacrificed on day 8. Each stomach was dissected along greater curvature. Reduction of ukertion score by 50 percent or more (≧50%) relative to vehicle control score values isconsidered significant.

The results indicate that, MIC-31 at 50, 25 and 12.5 mg/kg PO, and at 12.5 mg/kg I.P., caused a significant decrease (≧50%) in gastric ulceration relative to the vehicle control. As a positive control, Omeprazole (1 mg/kg) in combination with Clarithromycin (10 mg/kg), was given orally once daily for 7 consecutive days beginning with the first dose at one hour after Helicobacter pylori inoculation. The treatment resulted in a significant decrease (≧50%) in ulceration score relative to the vehicle-treated group. These results indicate that MIC-31 at 50, 25 and 12.5 mg/kg (P.O.) and at 12.5 mg/kg (I.P.), administered twice daily for 7 consecutive days starting one hour after Helicobacter pylori inoculation, afforded significant (≧50%) gastroprotective action against ulceration. Furthermore, although MIC-32 (geraniol) did not exhibit significant decrease in ulceration score, it certainly had some mild effect (13˜27% inhibition) as compared to the vehicle group. It is expected that when administered with higher dosage, genariol will demonstrate a better therapeutic result.

From the two experiments, it can be seen that in Example 1 the therapeutically effective dosage ranges from 30˜300 mg/kg, while in Example 2 the therapeutically effective dosage ranges from 6.25˜50 mg/kg. Rats were used as the animal model in Example 1, and mice were used in Example 2. According to the index of Human equivalent dosage, as listed in Table 6 (obtained from US FDA), the effective dosage to human is thus at least within the range of 0.5˜50 mg/kg. Note that the range of human effective dosage can be greater as long as it is within the reasonable health limit.

TABLE 6
Conversion of Animal Doses to Human Equivalent
Doses (HED) Based on Body Surface Area

| | | To convert animal | |
|---|---|---|---|
| | | dose in mg/kg to | |
| | To convert animal dose | HED^{a} in mg/kg, either: | |
| | in mg/kg to dose in | Divide | Multiply |
| | mg/m², multiply by km | animal dose | Animal |
| Species | below: | by: | dose by: |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |

Primates:

| | | | |
|---|---|---|---|
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

Although only gastric ulcer is evaluated in the examples, it is believed that Citronellol and its analogues/derivatives will also have the same therapeutic benefit on duodenal ulcer since the causes, symptoms and treatments for both types of ulcer are similar. Therefore, the scope of the present invention covers peptic ulcers in general.

Note that other Citronellol derivatives/analogues also have similar effect in the treatment or prevention of peptic ulcer. These Citronellol derivatives/analogues include Ciyronellone, Fema 2312, Fema 2317, Citronellyl isovalerate, Citronellyl benzene, Citronellyl anthranilate, Citronellyl nitrile, Citronellyl amine, Thiocitronellol, Citronellyl amide, 3,7-dimethyl-6-octenyl ethyl ether, 3,7-dimethyl-6-octenyl ropyl ether, 3,7-dimethyl-6-octenyl butyl ether, Citronellyl citronellol, Citronellyl Citronelloen, Generyl generiol, Generyl generone, Rarechem al bp 0330, Rarechem al bp 0340, Ethyl citronellate, Methyl citronellate. Therefore these Citronellol derivatives/analogues are also included in the scope of the present invention.

The forgoing embodiments are merely exemplary and are not to be construed as limiting the present invention. The present teachings can be readily applied to other types of apparatuses. The specification is intended to be illustrative, and not to limit the scope of the claims.

What is claimed is:

1. A composition for the treatment or prevention of peptic ulcer in mammals, said composition comprising isolated and purified Citronellol, Citronellol analogues and derivatives in an amount effective to treat or prevent a peptic ulcer and a pharmaceutical excipient,
wherein said Citronellol, Citronellol analogues and derivatives are selected from the group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof, and
wherein said peptic ulcer is induced by alcohol consumption, stress, use of aspirin and nonsteroidal anti-inflammatory medications or Helicobacter pylori infection.

2. (canceled)

3. (canceled)

4. The composition of claim 1, wherein said composition is administered orally or through intravenous or intraperitoneal injection.

5. The composition of claim 4, wherein said composition is prepared in powder, particle, capsule, tablet, injectable perfusion, oral solution, oral suspension, or other pharmaceutically available forms.

6. A pharmaceutical formulation for the treatment or prevention of peptic ulcer in mammals, said formulation comprising purified Citronellol, Citronellol analogues and derivatives in an amount effective to treat or prevent a peptic ulcer
wherein said Citronellol, Citronellol analogues and derivatives are selected from the group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof, and
wherein said peptic ulcer is induced by alcohol consumption, stress, use of aspirin and nonsteroidal anti-inflammatory medications or Helicobacter pylori infection.

7. The pharmaceutical formulation of claim 6, wherein said pharmaceutical formulation is delivered with a pharmaceutically acceptable carrier, diluent or excipient.

8. (canceled)

9. The pharmaceutical formulation of claim 6, wherein said effective amount is at least 0.5 mg/kg.

10. The pharmaceutical formulation of claim 6, wherein said effective amount ranges from 0.5 to 50 mg/kg.

11. A method for treating or preventing peptic ulcer in mammals, comprising: administering to a subject a composition comprising isolated and purified Citronellol, Citronellol analogues and derivatives in an amount effective to prevent a peptic ulcer
wherein said Citronellol, Citronellol analogues and derivatives are selected from the group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof, and
wherein said peptic ulcer is induced by alcohol consumption, stress, use of aspirin and nonsteroidal anti-inflammatory medications or Helicobacter pylori infection.

12. (canceled)

13. (canceled)

14. (canceled)

15. A pharmaceutical agent comprising pure Citronellol, Citronellol analogues and derivatives in an amount effective to prevent a peptic ulcer selected from a group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof, and a pharmaceutical excipient.

16. A diet supplement comprising an effective amount of isolated and purified Citronellol, Citronellol analogues and derivatives selected from a group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof, and a vehicle.

17. The diet supplement of claim 16, wherein said vehicle is a food or food ingredient.

18. A pharmaceutical formulation for the treatment or prevention of peptic ulcer in mammals, said formulation comprising an effective amount of isolated and purified Citronellol, Citronellol analogues and derivatives to prevent a peptic ulcer and a pharmaceutical excipient,
wherein said Citronellol, Citronellol analogues and derivatives are selected from the group consisting of: Citronellol, Geraniol, Citronellal, Citronellic acid, (s)-(+)-Citronellyl bromide, Citronellyl isobutryrate, Citronellyl acetate, Citronellyl propionate, Citronellyl formate, (R)-(−)-Citronellyl bromide, Citronellyl tiglate, (—)-β-Citronellol, and the combination thereof,
wherein said peptic ulcer is induced by alcohol consumption, stress, use of aspirin and nonsteroidal anti-inflammatory medications or Helicobacter pylori infection, and wherein said effective amount is at least 0.5 mg/per Kg of subject.
